(19) United States
(12) Patent Application Publication
(10) Pub. No.: US 2007/0260088 A1
(43) Pub. Date: Nov. 8, 2007

(54) Calcium Trifluoroacetate for Preparing Antiangiogenetic Medicaments

(76) Inventor: Rosa Gobbi, Fino Mornasco (IT)

Correspondence Address: YOUNG & THOMPSON, 745 SOUTH 23RD STREET, 2ND FLOOR, ARLINGTON, VA, 22202, US

(21) Appl. No.: 11/630,434

(22) PCT Filed: Jun. 17, 2005
(86) PCT No.: PCT/EP05/06533

§ 371 Date: Mar. 12, 2007

(30) Foreign Application Priority Data
Jul. 5, 2004 (EP) 04076932.5

(51) Int. Cl. C07C 53/21

(52) U.S. Cl. 562/605

(57) ABSTRACT

Calcium trifluoroacetate, known to have cytotoxic activity, is endowed With of antiangiogenetic activity and can be used for the therapy of atherosclerotic plaque, rheumatoid arthritis, psoriasis, diabetic retinopathy, rosacea, cheloids, metastasis.

The present invention relates to the use of calcium trifluoroacetate for the preparation of medicaments with antiangiogenetic effect.

### TECHNOLOGICAL BACKGROUND

Angiogenesis is a process leading to formation of new vessels, connected with blood circulation, through activation of vascular endothelial cells which are part of pre-existing capillaries and venules. In response to angiogenetic stimuli, the endothelial cells migrate into the perivascular space, where they proliferate and form the novel vessels.

In the adult, endothelial cells show very slow turnover, and angiogenesis is restricted to physiological conditions such as wound reparation and cyclic processes of the female reproductive system (ovulation, menstruation, implant and pregnancy). Physiological angiogenesis is strictly controlled, activated for short periods, only as far as tissue metabolic requests are concerned, then promptly inhibited.

Angiogenesis is also the “common denominator” of a number of pathological conditions in humans. It is in fact involved in the vascularization of the atherosclerotic plaque, promoting its instability and formation of thrombi, in the tissue damage deriving from rheumatoid arthritis and psoriasis, in the development of diabetic retinopathy, as well as in the development of solid tumors. In particular, it is established that a tumor cannot grow beyond a few millimetres in the absence of vascularization. Moreover, tumour angiogenesis promotes invasivity and metastatic diffusion of tumours.

EP 1 423 131 discloses that calcium trifluoroacetate and related calcium salts inhibit tumor growth in animal experimental models and exert in vitro cytotoxic activity on solid tumor cells.

### DISCLOSURE OF THE INVENTION

It has now been found that calcium trifluoroacetate and related salts disclosed in EP 1 423 131, in addition to cytotoxic and antitumor activity on solid tumors, have surprisingly marked antiangiogenetic activity.

The effect of calcium trifluoroacetate in angiogenesis was evidenced in a murine model of angiogenesis induced by a subcutaneous implant of Matrigel. Matrigel is liquid at 4° C. and solid at 37° C., so that a pro-angiogenetic stimulus can be mixed with Matrigel and inoculated. In this protocol, the selected angiogenetic factor was endothelial growth factor (VEGF), added to the Matrigel together with 16 U/ml heparin, as described in literature (Bussolati B, Altered angiogenesis and survival in human tumor-derived endothelial cells. FASEB J. 2003; Bussolati B, Vascular endothelial growth factor receptor-1 modulates vascular endothelial growth factor-mediated angiogenesis via nitric oxide. Am J Pathol. 2001).

Calcium trifluoroacetate was administered intraperitoneally 30 minutes before Matrigel, then on alternate days (day 2, 4, 6), at a dose of 2 or 4 μg/mouse (0.2 mg/Kg). After 7 days, the animals were killed and the Matrigel plugs were removed for histological examination. Angiogenesis was evaluated by computer assisted morphometric analysis of the percentage of area covered by vessels.

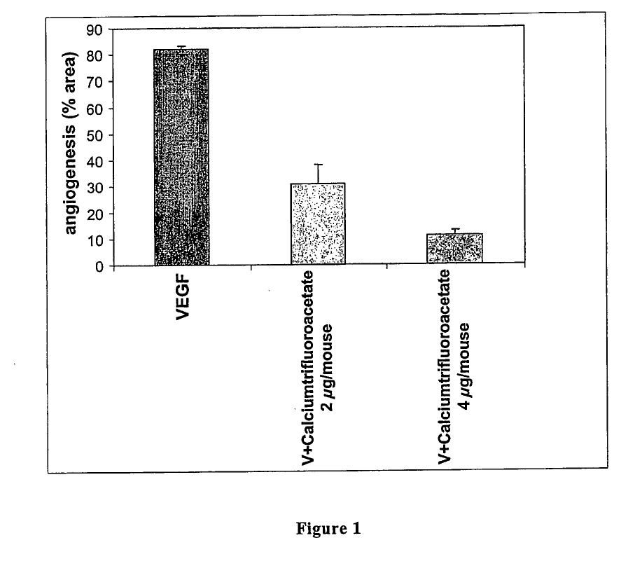

FIG. 1 shows the reduction of angiogenesis in mice treated with VEGF+calcium trifluoroacetate on alternate days at a dosage of 2 μg/mouse (n=4) and of 4 μg/mouse (n=8) compared with VEGF alone (n=8). P<0.001.

A marked angiogenesis was observed in the Matrigel plugs containing VEGF (40 ng/ml), as expected. The effect of VEGF was dramatically reduced in mice treated with calcium trifluoroacetate. This strong antiangiogenic effect could already be observed macroscopically and confirmed histologically. Morphometric analysis showed a reduction in angiogenesis above 40% with a dosage of 2 μg/mouse of calcium trifluoroacetate, and of 80% with a dosage of 4 μg/mouse.

Calcium trifluoroacetate has evident inhibitory action on endothelium proliferation. Moreover, calcium trifluoroacetate is able to block, at least partly, the effect of the usual endothelial growth factors on calcium entrance into the single endothelial cells.

These data suggest that calcium trifluoroacetate can inhibit the response of endothelial cells to stimulation by growth factors, likely acting on the message transmission through the calcium channels.

It is not intended, however, that the present invention be limited to this or any other mechanism of action.

Calcium trifluoroacetate can therefore be successfully used, in the form of suitable pharmaceutical compositions, for the treatment of pathologies in which the inhibition of angiogenesis is necessary or appropriate. Examples of said pathologies, as mentioned above, include atherosclerotic plaque, rheumatoid arthritis, psoriasis, diabetic retinopathy, rosacea, cheloids and other diseases or cutaneous inaesthetisms due to neovascolarization. Furthermore, calcium trifluoroacetate can be useful as antimetastatic agent to prevent the development of solid tumors.

For the envisaged therapeutical uses, calcium trifluoroacetate will be administered through the oral, topical, transdermal or parenteral (subcutaneous, intramuscular or intravenous) routes at dosages similar to those disclosed in EP 1 423 131, for example ranging form 20 to 100 mg/kg for the oral and parenteral routes, or at concentrations ranging from 2.5- to 10% for the topical formulations.

Calcium trifluoroacetate will optionally be combined with other active ingredients having complementary or anyway useful activity.

1. The use of calcium trifluoroacetate for the preparation of medicaments with antiangiogenetic effect.

2. The use as claimed in claim 1 for the preparation of medicaments for the treatment of atherosclerotic plaque, rheumatoid arthritis, psoriasis, diabetic retinopathy, rosacea, cheloids.

3. The use as claimed in claim 1 for the preparation of medicaments with activity on the growth of tumors mediated via angiogenesis.
